(19) United States
(12) Patent Application Publication
(10) Pub. No.: US 2004/0029261 A1
(43) Pub. Date: Feb. 12, 2004

(54) Methods for conducting assays and devices for use therein

(75) Inventor: Christopher Oldfield, East Lothian (GB)

Correspondence Address: HESLIN ROTHENBERG FARLEY & MESITI PC, 5 COLUMBIA CIRCLE, ALBANY, NY, 12203, US

(73) Assignee: Nanosphere Limited, Penicuik (GB)

(21) Appl. No.: 10/600,970

(22) Filed: Jun. 20, 2003

Related U.S. Application Data
(63) Continuation of application No. PCT/GB01/05666, filed on Dec. 20, 2001.

(30) Foreign Application Priority Data
Dec. 20, 2000 (GB) 0031049.0

(51) Int. Cl. C12M 1/34

(52) U.S. Cl. 435/287.2

(57) ABSTRACT

An apparatus for use in an assay to be conducted upon a fluid sample suspected of containing an analyte of interest, comprises a vessel for receiving a sample, and an element for capturing an analyte of interest. The element is insertable into the vessel for the purposes of the assay, and is coated on at least one surface with a specific binding partner for the analyte of interest. The element further having on a surface remote from the coated surface, structure adapted for manipulating the element for convenient analysis, whereby it is possible to detect and quantify microorganisms, particularly assay for infective agents such as bacteria, and viral particles and prions, or other particles that may be associated with a disease state, especially in humans.

### FIELD OF INVENTION

The present invention relates to assays useful in detecting and quantifying microorganisms, particularly assays for infective agents such as bacteria, and viral particles and prions, or other particles that may be associated with a disease state, especially in humans. Thus the invention is broadly applicable to a number of scientific fields involving detection and study of such agents, including microbiology, environmental sampling and monitoring, clinical chemistry, immunology, etc. The invention provides methods and devices for facilitating such assays, and kits for conducting such assays.

### BACKGROUND TO THE INVENTION

Many protocols exist for conducting assays to determine presence of an infective agent. These involve use of homogenous or heterogeneous systems, where these distinguish broadly between systems which are entirely liquid phase and those involving a solid phase. The present invention is applicable to heterogeneous systems.

In order to conduct an assay, it is necessary to introduce reagents that will interact with an analyte of interest in such a way that the presence or absence of the analyte can be detected and visualised. The methodology has become quite sophisticated, and there are both direct and indirect protocols available for qualitative or quantitative analysis. A chosen reagent may interact directly with the analyte of interest or with another reagent in the system known to react with the analyte of interest in a predictable way. The reagents may be labelled to facilitate the analysis. Immunoreagents may be radio-labelled, and detected by autoradiography, or visualised in other ways for example by binding with large molecules and detected optically by light scattering or absorbance techniques, such as nephelometry, turbidimetry etc. As these techniques are developed, there is evidence of increasing complexity, and there remains a need for reliable, yet simple and easy to use techniques by persons of relatively low skill levels, without the need to rely on sophisticated analytical instrumentation, or involving lengthy sample, and reagent, preparation steps.

Furthermore, whilst there are many techniques for detecting the presence of an analyte of interest, e.g. a pathogen, these are not necessarily capable of providing a quantitative analysis which is an essential requirement in many environmental health applications. Therefore, there is a need for an analysis technique that permits not only detection of pathogens but also enumeration thereof. Such a need arises in environmental monitoring, e.g. in assessing the quality of water supplies and comparing that quality against a predetermined standard e.g. for potable water supplies.

Accordingly, it is an object of this invention to provide improvements in methods of detecting infective agents, and to provide devices useful for such purposes. Particularly, a further object of the invention is to provide improvements in quantitative analysis of pathogens, and provide improvements in enumerating specific analytes of interest, e.g. specific antigens in a sample, which may contain a number of contaminants. A further object of the invention lies in providing an assay useful in environmental sampling and devices useful for such purposes.

### SUMMARY OF THE INVENTION

According to one aspect of the invention, there is provided a device for use in an assay to be conducted upon a fluid sample suspected of containing an analyte of interest, comprising a vessel for receiving a sample, and an element for capturing an analyte of interest, said element being insertable into said vessel for the purposes of the assay, and being coated on at least one surface with a specific binding partner for said analyte of interest, and said element further having on a surface remote from said coated surface means for manipulating the element, e.g. a projection or hook capable of being gripped by forceps or the like.

Preferably, the vessel has a closure permitting containment of sample during at least one stage of the assay.

The insertable element may be provided with means for facilitating its convenient location within the vessel at a desired position. Thus, in one embodiment, the vessel may be provided with means for providing an interference fit with said insertable element, such as shoulders adapted to receive a corresponding lip on said insertable element. Many alternative ways of achieving an interference fit between the vessel and the insertable element are possible, but due regard must be paid to the interest in achieving a simple and selectively releasable mechanism that permits ease of cleaning between assays and which will not tend to harbour contamination. A convenient form of vessel satisfying such needs is described hereinafter.

It may be desirable to provide retaining means for the insertable element to ensure that it is unlikely to be dislodged from the desired location within the vessel during the assay. This may take the form of a resilient biasing member, such as a spring. A coil spring for example may be conveniently located between a closure element, such as a screw cap, and the insertable element to urge the latter into engagement with support shoulders provided inside the vessel. An alternative possibility would be a lining sleeve adapted to closely fit against the vessel interior and abut the insertable element to restrain it from undesirable movement. A still further possibility would be to provide the closure element with a projection adapted to contact the insertable element when the latter and the closure element are properly located so that again the insertable element is restrained from undesirable movement during the assay. Other suitable means of locating the insertable element are described hereinafter.

The vessel may be a tubular body, of any convenient cross-section, but the smooth curvature of the inner wall provided by a tubular vessel of circular cross-section should be the easiest to clean. Thus the vessel may be a test tube, vial, or the like purpose-designed laboratory assay vessel. Alternatively, it may be a removable sampling tube arranged in a conduit system equipped with appropriate branch line and valve elements permitting fluid stream by-pass for sample collection using suitable conduit closure means such as taps for retaining a predetermined aliquot of sample within the tube.

The insertable element for capturing the analyte of interest would be typically designed to extend across the full interior width of the vessel. Such an element preferably comprises a foraminated surface to permit fluid flow therethrough. The said foraminated surface may extend across the entire width surface of the insertable element or may be confined to particular zones. The aperture size of the foramina in said foraminated surface would be designed with a view to avoiding clogging with particulate materials in the fluid to be assayed. In particular, it is desirable to permit free flow of particulates through the insertable element to avoid possible occlusion of surface bearing the specific binding partner for the analyte of interest. Thus, the insertable element should be designed to act as a macroporous support for the specific binding partner for the analyte of interest.

In one embodiment of the invention, a particular form for the insertable element, which offers some advantages for the proper and efficient conduct of the assay, is a substantially planar disk shape. This is preferably formed from square mesh grid, wherein one side of the mesh grid has said specific binding partner for the analyte of interest applied thereto, and the other has a projection facilitating manipulation of the insertable element to permit ease of insertion into the vessel and subsequent removal therefrom. Such a mesh may be divided into zones or sectors, each of such zones or sectors being coated with a different specific binding partner for correspondingly different analytes of interest. Alternatively, a stack of differently coated elements may be used, the respective elements in the stack being coated with a different specific binding partner for an analyte of interest. Either of these options would permit simultaneous determination of the content of such differing analytes of interest in a fluid sample in a single assay analyte-capture procedure.

The individual grid elements to be assembled into a stack, may be formed such as to permit convenient stacking, or provided with means for facilitating stack assembly, e.g. cradles or the like mechanical supports.

A variety of materials may be used for the vessel and its insertable element. Such will be selected for the assay on the basis of a number of factors including cost, inertness with respect to the possibility of contaminating or interfering with the desired progress of the assay, durability and ease of cleaning for re-use. Thus plastics, metals including alloys, ceramics and glasses can be contemplated.

The specific binding partner to be applied to a surface of the insertable element may be for example an antibody, and the antibody may be polyclonal or monoclonal, provided that in the context of the assay protocol that antibody exhibits the desired specificity for the target analyte, and does not exhibit non-specific binding to any other components of the sample to be assayed.

Typically, the fluids to be assayed would include water, milk, fruit juices, soft drinks, sewage sludges, biological fluids of animal or human origin, or washings from a surface such as a work surface or fruit or vegetables, inter alia.

According to another aspect of the invention there is provided an assay for enumerating analytes of interest, said assay comprising:

(a) providing a vessel capable of receiving an insertable element and a sample fluid;

(b) providing an element that is adapted to be inserted into said vessel, and that is coated on at least one surface with a specific binding partner for an analyte of interest, said element further having on a surface remote from said coated surface means for manipulating the element;

(c) introducing sample fluid to the vessel;

(d) permitting the sample fluid to contact the insertable element, and optionally agitating the sample, so that the insertable element will capture any analyte of interest by specific binding of the specific binding partner coated on said element therewith;

(e) removing the insertable element from the vessel and applying the surface coated with specific binding partner to a developer surface in a medium remote from the vessel to permit detection of binding events; and

(f) enumerating developed events indicative of specific binding between the specific binding partner and analyte of interest.

A variety of detection methods are available to permit counting of binding events captured upon the insertable element depending upon the labelling opportunities available for the sample containing the analyte of interest and its anticipated specific binding partner. In the case of an assay for particular micro-organisms the developer surface is desirably a non-liquid culture medium known to be favourable to proliferation of the target micro-organism. Thus, the insertable element may be applied to the surface of a gel media containing nutrients favourable to growth of the target micro-organism. The aforesaid means for manipulating the element facilitates the application of the element to a developer surface such as an agar plate with minimal risk of possibly contaminating the surface bearing the coating of specific binding partner for the analyte of interest. Ideally, light pressure is used to leave a slight impression in the agar. Where the element is formed from a mesh grid, the advantages of the regularity thereof in the subsequent colony counting stage will be apparent to those familiar with counting inoculated plates.

Subsequent incubation of the plate will permit any developing colonies of the target micro-organism(s) to be detected and counted after a suitable incubation period.

According to a further aspect of the invention there is provided a kit for conducting a quantitative assay upon a liquid sample, the kit comprising, a sample tube, a foraminated element insertable into the tube, and having coated on one surface thereof a specific binding partner for the analyte of interest, and on a surface remote from that coated surface a structure facilitating manipulation of the element e.g. by use of forceps which may be included in the kit. The kit may further comprise developers for visualising the binding events captured upon the removable element, and may include culture media and supports. Optionally, the removable element may be provided free of specific binding partner and the kit may include a plurality of specific binding partners for differing target analytes to be applied to the removable element by a user for the purposes of providing a custom assay device for a particular purpose.

### BRIEF DESCRIPTION OF THE DRAWINGS

The invention will now be further described by way of example with reference to the accompanying drawings in which,

FIG. 1A is a plan view of a lower surface of an insertable element, which is intended to act as a receptor for binding of analytes of interest in normal use of the element;

FIG. 1B is a vertical section through the plane of the element shown in FIG. 1A;

FIG. 1C is a plan view of an upper surface of the insertable element shown in FIG. 1A;

FIG. 1D is a plan view of a grid surface analogous to that shown in FIG. 1A, and schematically illustrating a bio-sensitive coating;

FIG. 2A is a section through a schematic representation of a vessel into which an element as shown in FIG. 1 is being inserted by means of a manipulation tool;

FIG. 2B is an end view of the jaws of the manipulating tool shown in FIG. 2A;

FIG. 2C is a section through the vessel and element as in FIG. 2A, but also illustrating schematically a closure cap for the vessel and retaining means for the element whereby the element is retained as placed and secured;

FIG. 3 provides three views of a culture vessel into which a plurality of elements which have been exposed to a sample for testing are inserted to be developed after being used to capture analytes; the uppermost view providing a section through such a vessel and a cooperating position alignment tool, whereby the elements are positively positioned in a desired position, as shown in plan in the middle view; and finally the so positioned elements are covered after removal of the alignment tool as shown in the final sectional view;

FIG. 4 shows views of a cradle device suitable for use in manipulation of insertable elements;

FIG. 5 shows the cradle being used with a tool to manipulate a plurality of insertable elements;

FIG. 6 shows use of a suitable tool adapted for use by a robotic actuator and “top hat” closure device to manipulate an insertable element or stack of insertable elements;

FIG. 7 shows the use of the top hat device of FIG. 6, and cradles to locate a stack of insertable elements in a tubular vessel; and

FIG. 8 illustrates schematically the dismantling of an assembly of insertable elements and cradles.

### MODES FOR CARRYING OUT THE INVENTION

The dimensions and materials of construction described in this example are illustrative of those appropriate for many uses of the invention but are provided by way of example and are not exhaustive of the possibilities available for utilisation of the invention.

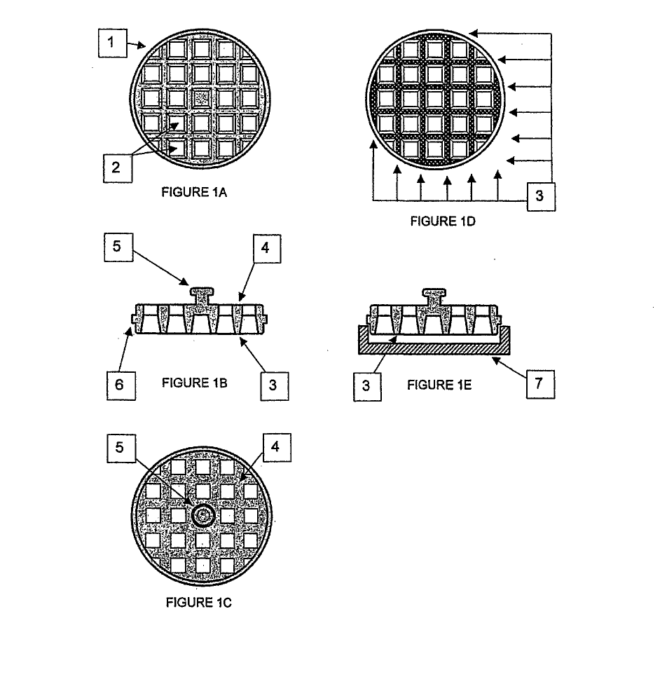

As shown in FIGS. 1A to 1D, an insertable element comprises a grid (hereafter “grid”). The grid is provided in the form of a circular disk and it incorporates an array of apertures 2. The grid thickness can be in the range 1 to 20 mm but is typically in the range 4 to 5 mm. The grid diameter may be in the range 10 mm diameter to 50 mm diameter, but is typically in the range 25 to 30 mm diameter. The apertures, for efficient through flow of the liquid test sample, are conveniently square in cross section and are typically in the range 3 to 4 mm side length.

The underside of the grid 3 is the surface which is first presented to the liquid test sample and the apertures on this side incorporate lead-in chamfers to assist channelling the liquid through the apertures. The underside 3 is a flat lattice shaped area upon which is deposited the layer (or layers) of bio-sensitive coating. The topside of the grid 4 appears as a plain grid of square apertures and optionally incorporates a projection, a central bollard 5 which can be used as a grid handling feature. On the outer circumference of the grid is an optional rectangular section lip 6 which provides an alternative handling feature. This lip will typically be less than 1 mm in radial width and typically be in the range 1 to 2 mm in height. A further benefit of this lip is that it can be used to prevent contamination of the sensitised grid surface 3. This is illustrated in FIG. 1E where a grid holder 7 contacts the lip but not the sensitised surface 3. Various grid handling methods will be described which rely on one or the other of the handling features 5 and 6, but nothing precludes both these handling features being present at the same time, conveniently allowing the use of a “standardised” grid which has more than one possible-handling method.

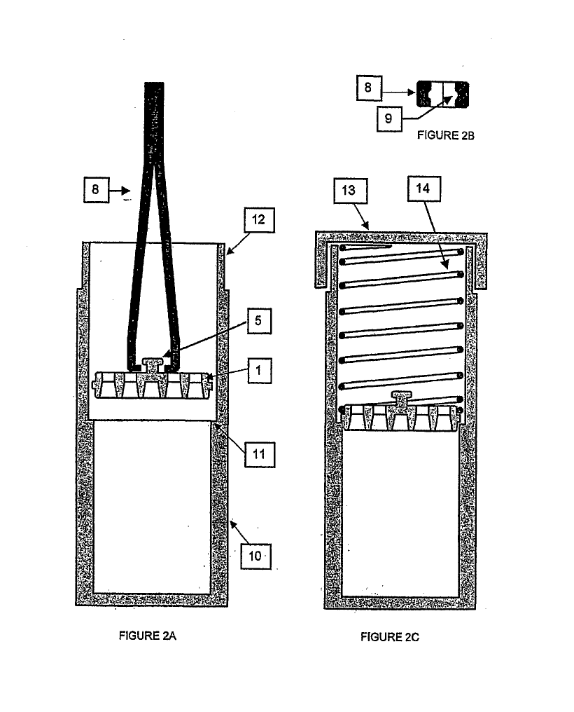

The first, and simplest, handling method is described using the bollard 5 and is illustrated in FIGS. 2A to 2C. This method is a manual method of grid handling. A hand-held tool 8 is provided in the form of a pair of forceps where the free ends of the forceps are geometrically configured as jaws 9 to grip around the shape of the bollard 5 (see FIG. 2B, an end view of the forceps). The forceps action will conveniently be normally biased open, requiring simply finger pressure to hold them shut and lift the grid, but an inverse action is also possible. The grid 1 is required to be loaded into a vial 10 within which is contained the liquid test sample. This vial may incorporate a threaded area 12 for the fastening of a lid 13. A grid picked up by the forceps 8 can be inserted into the vial 10 and lowered on to an internal shoulder 11 within the vial. The grid is released and a coil spring 14 is inserted such that when the vial lid 13 is assembled, the grid 1 is held captive to the shoulder 11 by the compression of the spring 14. The vial may then be inverted or shaken without dislodgement of the grid.

The vial is non proprietary and preferably is a standard plastic vial incorporating a standard threaded lid and widely available from laboratory suppliers.

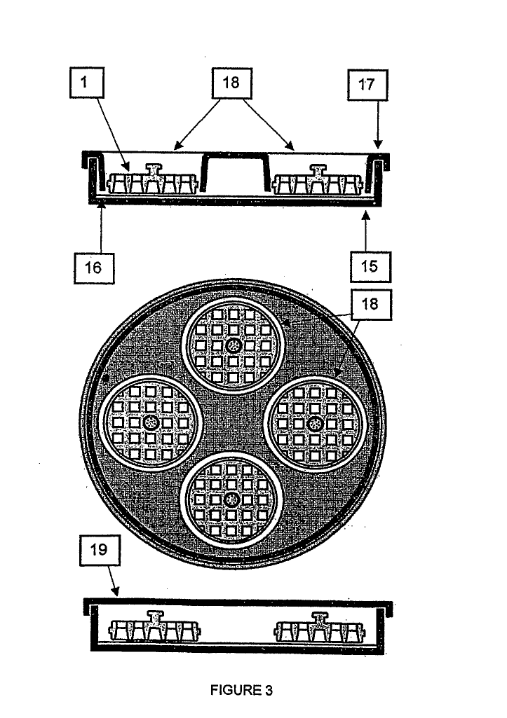

A further use of the bollard 5 is to assist in manually loading the grid 1 on to an agar culture plate where incubation and multiplication of the bio-material captured on the bio-sensitive layer 7 can take place. FIG. 3 illustrates this process. A circular plate 15 contains agar culture medium 16. This is a standard industrial plate which is suitable for accommodating 4 grids simultaneously. A lid 17 with loading alignment apertures 18 which have internal skirts which extend downwards close to the agar layer ensures that the optimum number of grids is loaded. Once the grids are in place, this alignment lid is replaced by a non proprietary sealing lid 19. Without the alignment lid, improper manual positioning may restrict the number of grids per plate, or inhibit rapid sample processing by the need to take extra time to carefully manually position the grids.

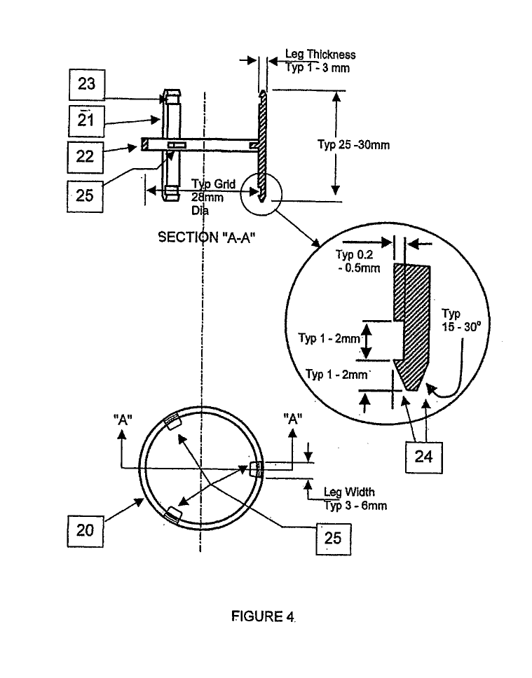

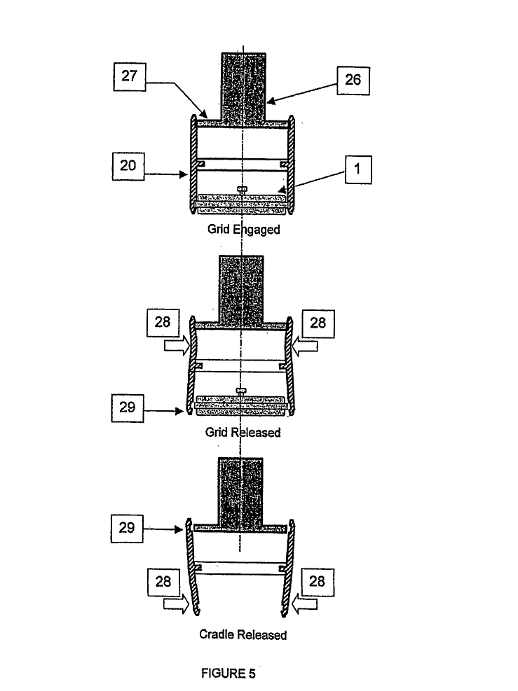

A further method of handling the grid is described by introducing a cradle component 20 as in FIG. 4. The cradle comprises three legs 21, a supporting circular band 22, grooves 23 at the end of each leg, lead in chamfers 24 on the end of each leg, lugs 25 on the inside of each leg at a mid point of each leg. The cradle may be picked up by a disk shaped handling tool 26 as in FIG. 5 by snapping the tool into the upper set of grooves. Ease of engagement is aided by the chamfers on the upper end of each leg. The method of attaching the handling tool to the cradle or the grid to the cradle is therefore easily achieved by a snap-fit action. The groove 23 dimensions will correspond to the dimensions of the lip 6 on the grid, but have a slightly larger width (for example, where the lip width is 1.5 mm, the groove width may be 1.6 mm) and the groove depth will be shallow such that removal of the handling tool 26 from the cradle 20, or the grid 1 from the cradle 20 can be effected by a bending action of the leg 21 simultaneously on each leg.

FIG. 5 illustrates the method of removal of the grid 1 from the cradle 20 or the handling tool 26 from the cradle 20. To release the grid 1, an inward radial side force is applied to each upper limb 28 of the leg 21 approximately mid-way on the upper limb. This causes the legs to deform and pivot about the circular band 22 causing the lower limbs 29 to deform in an outward radial direction thereby releasing the grid. The means of applying the side force is non proprietary and can deploy mechanisms such as an internal expanding mechanical collet, a series of radially acting pneumatic actuators or an expanding pneumatic torus. The method of releasing the handling tool 26 uses a similar method, whereby the side compressive force is applied on the lower legs 29 of the cradle.

The geometry of the cradle will optimally have symmetry about the circular band, whereby the upper and lower legs have identical geometry to avoid the need for positioning of the cradle in a preferred orientation prior to handling. The discrete lugs 25 shown may optionally be configured as a continuous flange ring. The cradle 20 may optionally be configured with four or more legs 21.

The cradle may be manufactured by an injection moulding process using a wide range of plastic materials such as polystyrene, polyacetal, polyamide, ABS, polycarbonate or polysulfone. Optimum mechanical properties relating to the “snap action” of the legs may be aided by fillers such as glass bead or glass fibre.

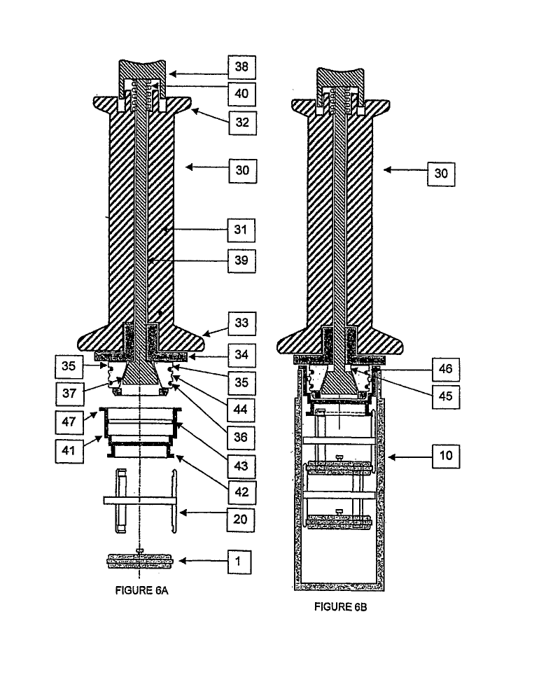

A further method of handling the grid is described in FIG. 6. In this example, a tool 30 comprises a handle 31, which is approximately cylindrical, with flared ends 32, 33 to ensure a good hand grip. The lower flared end 33 can also act as a splash guard. This end of the handle incorporates a body 34 which holds an expanding jaw mechanism comprising jaws 35, jaw coil springs 36 and moving cone 37. The jaws slide radially in geometrically matching slots in the body 34 and are biased in a normally inward direction by the single coil springs 36 which hold the jaws radially inward against the cone 37. The cone 37 can be moved downward by pressing a pad 38 which is attached to a shaft 39 which passes through a hole along the axis of handle 31. As the cone 37 is moved downward, the jaws 35 move radially inward. The cone 37 is biased normally upwards by a coil spring 40 acting on the underside of pad 38.

The function of this handling tool 30 is to engage with, and lift, a “top hat” 41 component which in turn incorporates a lower flange 42 suitable for snap engagement with cradle 20 which in turn snap engages with grid 1. The primary engagement method of the jaws is by employing friction and the materials of construction reflect this, but optionally, this can be mechanically aided by employing an internal circular lip 43 in the top hat which corresponds to grooves 44 in the jaws.

When the handle is gripped with the thumb towards the upper end of the handle, it is an easy action of the thumb to press the pad 38. This whole arrangement provides a manual grid handling tool which is simple to operate and is positive and robust in action. The action is illustrated by comparing FIGS. 6A and 6B. In FIG. 6A the cone is in fully retracted state, where the upper face of the cone is held against the lower face of body 34 under the action of spring 40. In FIG. 6B the cone has been lowered by pressing pad 38 downwards to create gap 45 and the jaws 35 have retracted to create gap 46 which allows easy engagement with or disengagement from the top hat 41.

Having regard to the function of the jaws, due care should be taken in selecting the material of construction thereof. In a preferred construction, each jaw 35 is a two part fabrication where the outer (gripping) contact area is made of polyurethane, or hard rubber for high friction property and the inner sliding section including the contact area with the moving cone is made of low friction material such as PTFE or polyacetal. The cone and shaft preferably will be stainless steel. The coil springs 36 and 40, will preferably be made from corrosion resistant spring steel wire. The preferred material for the other components of the tool 30 will be plastic, with polyacetal being a suitable common choice.

An important benefit of this arrangement is the function of the top hat device 41. This component provides an alternative to the spring 14 and spacer 11 (shoulder) arrangement previously described in FIGS. 2A and 2C. The top hat 41 incorporates a flange 47 which engages on the open end of the vial 10 and is clamped in place when the lid 13 is assembled. The cradle and grid are therefore positively located relative to the lid end of the vial allowing the vial to be inverted or shaken without dislodgement of the grid. The top hat also isolates the liquid test sample from the handling tool when the lid is removed, thereby minimising the cleaning requirements of the handling tool. The top hat becomes a consumable item and may be manufactured by an injection moulding process using a wide range of plastic materials such as polyethylene, polypropylene, polystyrene, polyacetal, polyamide, abs, polycarbonate or polysulfone.

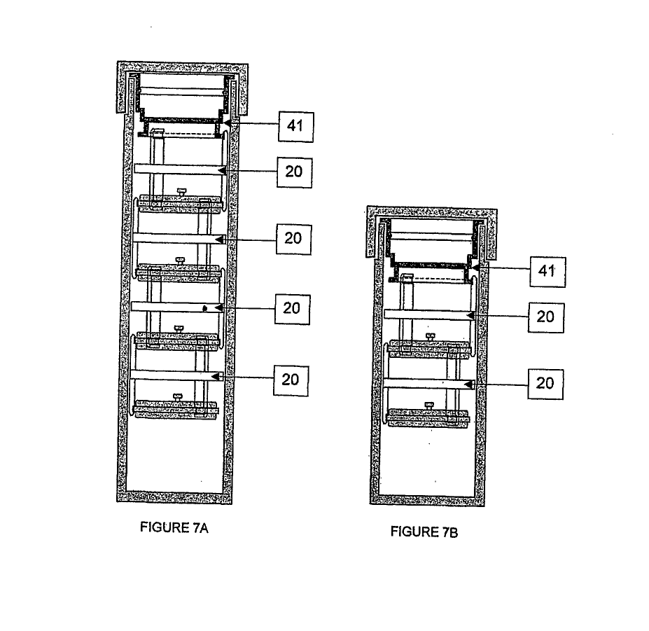

A further benefit of this arrangement is shown in FIGS. 7A and 7B. This illustrates that multiple grids can be loaded in a standard vial using only two components i.e. the top hat 41 and the cradle 20 and these components positively locate the stack relative to the top of the vial regardless of the number of grids in the stack. FIG. 7A illustrates a four-up grid stack, FIG. 7B a two-up grid stack. The number of grids is limited, in practice, only by the length of the vial and the stability of the grid stack relative to the gripper end of the handling tool. A stack of at least six grids is readily achievable by this method. Each grid 1 and each cradle 20 is picked up by an identical snap-fit action, the only additional requirement being that the tool 30 should be rotated between each pick up operation to ensure that the legs 21 of adjacent cradles do not clash.

A further benefit of this arrangement is that it can be deployed as part of an automated handling system. The handle 30 can be attached to, for example, a robotic manipulator arm and the actuation of pad 38 can be effected by a pneumatic cylinder. The manipulator arm provides capability to a) pick up the top hat 41, b) engage a cradle 20 with the top hat 41, c) engage a grid 1 on a cradle 20, d) engage a stack of cradles andgrids, e) load a stack of cradles and grids into a vial, f) unload a stack of cradles and grids from a vial, g) take the stack of cradles and grids to a grid release tool, h) take the stack of cradles and grids to other functional stations in the workspace such as a wash station, a drying station, a scanning station, a waste disposal station.

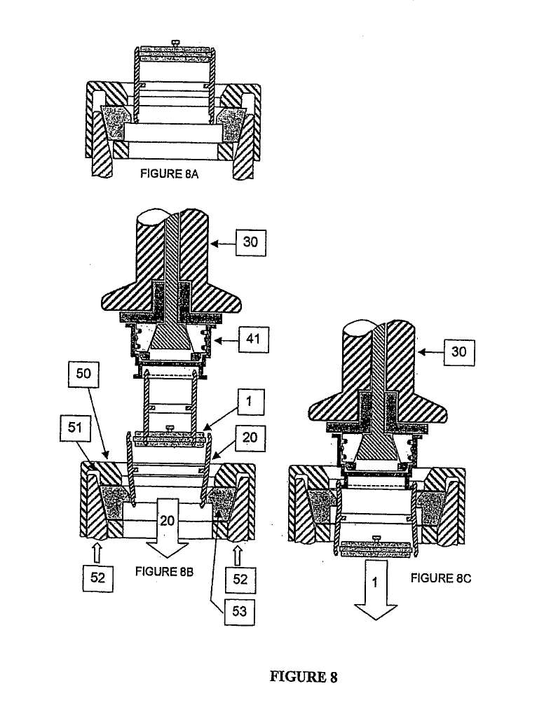

FIG. 8B illustrates a method for releasing a cradle 20 from a grid 1 using a non-proprietary collet mechanism. A release tool 50 is shown as an expanding internal collet assembly comprising a body 51, a collet actuator 52 whereby upward movement causes collet jaws 53 to move radially inwards. This upward movement can be effected, for example, by rotating a collar which is coupled to the collet actuator 52 by a threaded interface. The handling tool 30 is lowered into the release tool as shown in FIG. 8A where the collet jaws are fully open allowing free passage of the cradle 20. Tool 30 continues to lower as in FIG. 8B such that the lower ends of the lower legs of cradle 20 are aligned with the contact jaws 53 of the collet. Collet actuator 52 is moved upwards (see arrows), causing jaws 53 to extend inwards thereby releasing cradle 20 from grid 1. Tool 30 is then withdrawn from the release tool with the cradle 20 still held in the collet jaws 53. The jaws 53 are opened and the cradle 20 falls under gravity into, for example, a waste disposal chute. The situation in FIG. 8B now exists.

FIG. 8C illustrates a method for releasing a grid 1 from a cradle 20. Tool 30 is lowered into the release tool such that the mid point of the respective upper legs of cradle 20 are aligned with the contact jaws of the collet. Collet actuator 52 is moved upwards (see arrows), causing jaws 53 to extend inwards and release grid 1 which falls under gravity into, for example, a waste disposal chute. At this point in the sequence the remaining cradle is still attached to the top hat 41, but there is no need to detach this cradle from the top hat, the combined cradle and top hat pair are released (for example, into a waste disposal chute) by releasing handling tool 30 from the top hat as previously described.

The grid release tool can be mounted at a convenient location within the robotic manipulator workspace. The manipulator arm can bring the handling tool 30 and insert it into the grid release tool. The grid release tool can then be operated by an electric or pneumatic actuator. Removal and re-assembly of vial lids can also be effected automatically using a non proprietary rotary gripper device.

Mode of Performance of an Assay Using a Kit of the Invention

The following outlines one way of realising the invention with respect to the construction and method of use for a kit for the selective enumeration of micro-organisms (e.g. quantitative E. Coli detection kit) virions or prions in liquid samples. Such a kit, basically consists of a tube containing a removable disc-shaped element comprising a wide-mesh grid adapted to pass particulates but capture the analyte of interest. The ability to capture analyte of interest is accomplished by coating the element on one surface with a specific binding partner for the analyte of interest e.g. antibody that tightly and selectively binds the micro-organism or prion of interest. The sample is added to the tube, which is then capped and agitated for a sufficient length of time to permit substantially all instances of that target analyte in the sample to bind to the coated surface of the element.

The element is then removed from the tube and developed. There are several ways to do this, but one straightforward approach is to culture the captured micro-organisms as described in the Example on an agar plate. Alternatively, the removable element may be immersed in media and the analysis conducted instrumentally, e.g. by observing absorbance, or fluorescence.

Such an arrangement as described hereinbefore is ideal for the enumeration of micro-organisms or virions or prions in liquid samples such as water, waste waters, sewage sludges, dairy fluid products, e.g. milk, and biological fluids, e.g. blood, urine.

In one typical use of the invention, bacteria can be quantitatively immobilised on a square mesh-grid suitably supported in the sampling tube. The mesh size of the grid is selected such that it permits large particulate material to pass through, so that the active surface of the grid supporting a specific binding partner for the analyte of interest should not become occluded by fouling with such particulate material.

By adopting a grid structure in the removable element bearing the specific binding partner for the analyte of interest, a non-liquid, soft surface culture medium can be inoculated by pressing of the appropriate surface of the grid upon the exposed surface of the medium. This results in a grid pattern of inoculation, which subsequently facilitates counting either by the human eye, or by an instrumental technique.

A particular advantage of the invention lies in the fact that relatively large volumes of sample can be treated whilst retaining the ability to achieve a high sensitivity count. In modifications, the sampling tube can be adapted to receive a flow through of the predetermined sample volume, either in a single pass or in repetitive cycles to maximise the probability of achieving the desired specific binding and a more accurate count.

The accuracy of the proposed system is optimised by judicious selection of the specific binding partner e.g. by raising an antibody against a rationally designed antigen having regard to the intended application.

The antibody may be polyclonal or monoclonal in origin. The grid need not carry a single antibody. The grid could be divided into quadrants or sectors, each carrying a different antibody. The grid would be marked for reference, e.g. the underside could carry a raised pin at “12 o'clock”, which will make a corresponding impression on the agar plate.

The tube may contain more than one grid.

Method of Use

1 The unit may be supplied assembled and sterile. The screw cap is removed and the sample added. The screw cap is replaced tightly. The unit is clipped on a vertically rotating wheel (ca. 20 rpm), which allows the sample to mix thoroughly in the tube. Optionally the tube can be shaken gently by hand or agitated in any other convenient way permit the whole sample to pass over the surface of the element carrying the specific binding partner(s) for the analyte(s) of interest. Sufficient mixing time must be allowed for all cognate bacteria to bind to the grid via the cognate antibody.

2 The liquid sample and the element are separated and the grid is rinsed with a suitable rinsing solution (for say 5 min). This step is repeated. This rinsing step can be achieved by either decanting the sample and substituting rinse solution, or the element can be removed from the tube for rinsing.

Development of the Grid

(a) By Culturing the Grid

Using the forceps, the element is manipulated to present the grid to an agar plate surface and it is pressed down gently for good contact between the grid and the agar. The plate is incubated at the required temperature for 2 h. After this time the grid is removed and discarded. A firm impression of the grid should be seen in the agar. The plate is then incubated overnight.

It will be recognised that the approach can be used in conjunction with techniques for resuscitation of sub-lethally injured cells.

Colonies that appear on the square grid matrix are easily counted by eye or using standard digital imaging equipment.

A particular advantage of this approach, using a culture method of visualising the binding events that have occurred on the grid is that a viable cell count is obtained. This can be enhanced by the aforesaid resuscitation techniques.

(b) By Immunodetection

Alternatively, the grid can be developed directly by standard immunodetection techniques. The plate is incubated in a solution containing the cognate antibody and rinsed thoroughly. The plate is then developed by treatment with a fluorescence-labelled secondary antibody. The grid can be viewed under a fluorescence microscope, or appropriate digital imaging equipment.

It is possible that the element could be placed in a solution of matching refractive index, which would render the element invisible to an external viewer.

If the grid carries more than one antibody, it can be developed with secondary antibodies that fluoresce with different colours or exhibiting differing absorbances.

Alternatively the secondary antibody can be radiolabelled and the grid can be developed by autoradiography. The photographic film can be viewed directly or using digital imaging equipment.

Industrial Utility

The invention is useful for detecting and quantifying microorganisms and can be used in testing potability of water for example, or in screening for infective agents such as bacteria, and viral particles and prions, or other particles that may be associated with a disease state, especially in humans. Thus the invention is broadly applicable to a number of scientific fields involving detection and study of such agents, including microbiology, environmental sampling and monitoring, clinical chemistry, immunology, etc.

1. A foraminated element for use in an assay for an analyte of interest, said element being of a substantially planar configuration and having an obverse face and a reverse face, the obverse face having applied thereto a specific binding partner for the analyte of interest, and the reverse face having a projection extending out of the plane of the element to facilitate manipulation of the element.

2. An element according to claim 1, wherein the foraminated element is a substantially planar disk shape.

3. An element according to claim 1, wherein the projection is a bollard positioned centrally on the reverse face.

4. An element according to claim 1, wherein the projection is a hook.

5. An element according to claim 1, wherein the element comprises a grid of substantially uniform foraminations extending across the width of the element.

6. An element according to claim 5, wherein the grid comprises a square mesh defining a regular array of apertures.

7. An element according to claim 5, wherein the grid thickness lies in the range of from 1 to 20 mm.

8. An element according to claim 5, wherein the grid thickness lies in the range of from 4 to 5 mm.

9. An element according to claim 5, wherein the grid is provided in the form of a disk the diameter of which lies in the range of from 10 to 50 mm.

10. An element according to claim 5, wherein the grid is provided in the form of a disk the diameter of which lies in the range of from 25 to 30 mm.

11. An element according to claim 5, wherein the foraminations are square apertures having an aperture size defined by an aperture side length in the range of from 3 to 4 mm.

12. An element according to claim 1, wherein a peripheral lip is provided around the element, said lip having a height in the range of from 1 to 2 mm.

13. An element according to claim 1, wherein the obverse face of the element is a flat lattice-shaped area upon which is deposited a layer of a bio-sensitive coating.

14. An element according to claim 1, wherein the obverse face of the element is coated with an antibody.

15. An element according to claim 14, wherein the antibody is a monoclonal antibody.

16. An element according to claim 1, wherein the projection is adapted for manipulation using forceps.

17. An element according to claim 12, wherein the element is adapted for manipulation by an automatic handling system.

18. Apparatus for use in an assay to be conducted upon a fluid sample suspected of containing an analyte of interest, comprising a vessel for receiving a sample, and an element for capturing an analyte of interest, said element being insertable into said vessel for the purposes of the assay, wherein said element is a foraminated element having a substantially planar configuration and having an obverse face and a reverse face, the obverse face having applied thereto a specific binding partner for the analyte of interest, and the reverse face having a projection extending out of a plane of the element to facilitate manipulation of the element.

19. Apparatus according to claim 18, wherein the foraminated element has a peripheral lip provided around the element, said lip having a height in the range of from 1 to 2 mm.

20. Apparatus according to claim 19, further comprising a cradle for manipulating the element, said cradle comprising a plurality of legs attached to a support member in a manner such that said legs have upper and lower extremities projecting out of the plane of the support member, and capable of limited free-play movement about a point of support, in a radial direction with respect to a notional centre of said plane, whereby a compressive force on an upper extremity of a leg provokes an outward displacement of the corresponding lower extremity of said leg, and each of said legs has a groove at its lower extremity for engagement of the lip of said foraminated element by an interference fit, enabling the legs to be used to support or manipulate such an element.

21. Apparatus according to claim 18, wherein a plurality of differently coated elements are presented as a stack for insertion into the vessel, the respective elements in the stack being coated with a different specific binding partner for an analyte of interest.

22. Apparatus according to claim 18, wherein the foraminated element is provided with means for facilitating its convenient location within the vessel at a desired position.

23. Apparatus according to claim 22, wherein retaining means are provided for the foraminated element to retain same at the desired location within the vessel during use.

24. Apparatus according to claim 23, wherein said retaining means is a resilient element.

25. Apparatus according to claim 23, wherein said retaining means is a compression spring.

26. Apparatus according to claim 20, wherein the support member comprises a circular band and three legs are arranged, mutually equi-spaced, around a circumference of the band.

27. Apparatus according to claim 18, wherein said vessel is a conduit or chamber in a continuous flow system, and said foraminated element is insertable into said conduit or chamber such that fluid suspected of containing an analyte of interest contacts said foraminated element by flow therethrough.

28. Apparatus according to claim 18, wherein the specific binding partner is an antibody.

29. An assay for enumerating an analyte of interest, said assay comprising:
(a) providing a vessel capable of receiving a foraminated element and a sample fluid;
(b) providing a foraminated element that is adapted to be inserted into said vessel, wherein said element has a substantially planar configuration and has an obverse face and a reverse face, the obverse face having applied thereto a specific binding partner for the analyte of interest, and the reverse face having a projection extending out of a plane of the element to facilitate manipulation of the element;
(c) introducing sample fluid to the vessel;
(d) permitting the sample fluid to contact the foraminated element, and optionally agitating the sample, so that the foraminated element will capture any analyte of interest by specific binding of the specific binding partner thereto;
(e) removing the foraminated element from the vessel and applying the obverse face to a developer surface in a medium remote from the vessel to permit detection of binding events; and
(f) enumerating developed events indicative of specific binding between the specific binding partner and analyte of interest.

30. An assay according to claim 29, wherein the element is as claimed in claim 2.

31. An assay according to claim 29, wherein apparatus as claimed in claim 18 is used to capture an analyte of interest from a sample fluid.

32. An assay according to claim 29, wherein presence of an analyte of interest is determined by removing the foraminated element from the vessel and contacting the obverse face of the foraminated element with the surface of a solid nutrient medium, and incubating the contacted nutrient medium in order to facilitate culture development.

33. An assay according to claim 32, wherein the nutrient medium is an agar plate.

34. An assay according to claim 29, wherein, said vessel is a conduit or chamber in a continuous flow system, and said foraminated element is insertable into said conduit or chamber such that fluid suspected of containing an analyte of interest contacts said foraminated element by flow therethrough.

35. An assay according to claim 29, wherein the specific binding partner is an antibody.

36. An assay according to claim 35, wherein the antibody is a monoclonal antibody.

37. A kit for conducting a quantitative assay upon a liquid sample, the kit comprising, a sample tube, a foraminated element insertable into the tube, wherein said foraminated element has a substantially planar configuration and has an obverse face and a reverse face, the obverse face having applied thereto a specific binding partner for an analyte of interest, and the reverse face having a projection extending out of a plane of the element to facilitate manipulation of the element.

38. A kit according to claim 37, wherein the element is an element as claimed in claim 2.

39. A kit according to claim 37, wherein a cradle is provided for mounting and manipulating foraminated elements.

40. A kit according to claim 39, wherein said cradle comprises a plurality of legs attached to a support member in a manner such that said legs have upper and lower extremities projecting out of a plane of the support member, and capable of limited free-play movement about a point of support, in a radial direction with respect to a notional centre of said plane, whereby a compressive force on an upper extremity of a leg provokes an outward displacement of a corresponding lower extremity of said leg.

41. A kit according to claim 40, wherein the support member comprises a circular band and three legs are arranged, mutually equi-spaced, around a circumference of the band.

42. A kit according to claim 40, wherein the legs have grooves near the extremities thereof arranged for engagement of an edge of the element, by an interference fit, enabling the legs to be used to support or manipulate such an element.

43. A kit according to claim 42, wherein the foraminated element is adapted to engage with the cradle for manipulation thereby by provision of a peripheral lip adapted to “snap-fit” into the grooves of the said legs of the cradle.

44. A kit for conducting a quantitative assay upon a liquid sample, the kit comprising, a sample tube, a foraminated element insertable into the tube, wherein said foraminated element has a substantially planar configuration and has an obverse face and a reverse face, the obverse face having a flat lattice-shaped area for receiving a layer of material sensitive to an analyte of interest, the reverse face having a projection extending out of a plane of the element to facilitate manipulation of the element, and at least one reagent container providing therein a material to be applied to said flat lattice-shaped area for detecting the analyte of interest.

45. A kit according to claim 44, wherein the material to be applied to said flat lattice-shaped area is an antibody.
